(19) United States
(12) Patent Application Publication
(10) Pub. No.: US 2019/0233355 A1
(43) Pub. Date: Aug. 1, 2019

(54) SYSTEMS AND METHODS FOR MAKING ETHANOL PRODUCTS

(71) Applicant: Global Spirits Filtration Company, LLC, Mason City, IA (US)

(72) Inventor: Randy Wenthold, Menahga, MN (US)

(21) Appl. No.: 16/261,129

(22) Filed: Jan. 29, 2019

Related U.S. Application Data
(60) Provisional application No. 62/623,676, filed on Jan. 30, 2018.

(51) Int. Cl. C07C 29/84; C07C 31/08

(52) U.S. Cl. CPC C07C 29/84; C07C 31/08

(57) ABSTRACT

A method for producing an ethanol product includes obtaining a condensed distillate including ethanol at a concentration greater than 30% by volume and contacting the condensed distillate with an adsorbent polymer resin. The adsorbent polymer resin includes a polystyrene divinylbenzene copolymer. A surface of the adsorbent polymer resin is hydrophobic.

Another method for producing a purified ethanol product includes obtaining a vapor phase distillate including ethanol at a concentration greater than 30% by volume, contacting the vapor phase distillate with an adsorbent polymer resin, and condensing the vapor phase distillate to produce the purified ethanol product. The adsorbent polymer resin includes a polystyrene divinylbenzene copolymer. A surface of the adsorbent polymer resin is hydrophobic.

### CROSS-REFERENCE TO RELATED APPLICATION

This application claims priority to U.S. Provisional Application No. 62/623,676 filed Jan. 30, 2018, which is herein incorporated by reference in its entirety.

### TECHNICAL FIELD

The present disclosure relates to producing ethanol products. More specifically, the disclosure relates to methods and systems for purifying ethanol products.

### BACKGROUND

Certain products including ethanol can be produced from the distillation of the fermentation products of various plant and plant-derived materials, such as fruits, tubers, grains, honey, and sugar cane. Ethanol products may have many uses, such as for beverages or for internal or external medical formulations, for example. The ethanol products can include a variety of organic compounds in addition to the ethanol. Much of the character of the ethanol product can arise from the colors, flavors, and aromas of these organic compounds. However, some of the organic compounds can add colors, flavors, and aromas that are not desired in the final ethanol product. In some instances, it may be desirable to purify the ethanol product by removing some or substantially all of these organic compounds from the ethanol product.

### SUMMARY

Example 1 is a method for producing an ethanol product. The method includes obtaining a condensed distillate including ethanol at a concentration greater than 30% by volume and contacting the condensed distillate with an adsorbent polymer resin. The adsorbent polymer resin includes a polystyrene divinylbenzene copolymer. A surface of the adsorbent polymer resin is hydrophobic.

Example 2 is the method of Example 1, wherein contacting the condensed distillate with the adsorbent polymer resin includes flowing the condensed distillate through a vessel containing the adsorbent polymer resin.

Example 3 is the method of either of Examples 1 or 2, further including regenerating the adsorbent polymer resin.

Example 4 is the method of Example 3, wherein regenerating the adsorbent polymer resin includes contacting the adsorbent polymer resin with at least one of: steam and sodium hydroxide.

Example 5 is the method of any of Examples 1-4, wherein the concentration of the ethanol in the condensed distillate is between 30% and 95%.

Example 6 is the method of Example 5, wherein concentration of the ethanol in the condensed distillate is between 80% and 95%.

Example 7 is the method of any of Examples 1-6, wherein the adsorbent polymer resin is a macroporous polymer resin.

Example 8 is the method of Example 7, wherein the macroporous polymer resin includes at least one of: Dowex™ Optipore™ L493 adsorbent resin and Amberlite™ FPX66 adsorbent resin.

Example 9 is the method of Example 8, wherein the macroporous polymer resin consists of the Dowex™ Optipore™ L493 adsorbent resin.

Example 10 is a method for producing an ethanol product. The method includes obtaining a condensed distillate including ethanol at a concentration greater than 30% by volume and contacting the condensed distillate with a plurality of adsorbent materials. The plurality of adsorbent materials includes an activated carbon and a polymer resin. The polymer resin includes a polystyrene divinylbenzene copolymer. A surface of the polymer resin is hydrophobic.

Example 11 is the method of Example 10, wherein contacting the condensed distillate with the plurality of adsorbent materials includes flowing the condensed distillate through at least one vessel containing at least one of the plurality of adsorbent materials.

Example 12 is the method of either of Examples 10 or 11, wherein contacting the condensed distillate with the plurality of adsorbent materials includes contacting the condensed distillate with the activated carbon before contacting the condensed distillate with the polymer resin.

Example 13 is the method of any of Examples 10-12, wherein the plurality of adsorbent materials consists of the activated carbon and the polymer resin.

Example 14 is the method of any of Examples 10-13, wherein the polymer resin is between 1 wt. % and 99 wt. % of the plurality of adsorbent materials.

Example 15 is the method of any of Examples 10-14, wherein the concentration of the ethanol in the condensed distillate is between 30% and 95%.

Example 16 is the method of any of Examples 10-15, wherein the polymer resin is a macroporous polymer resin.

Example 17 is the method of Example 16, wherein the macroporous polymer resin includes at least one of: Dowex™ Optipore™ L493 adsorbent resin and Amberlite™ FPX66 adsorbent resin.

Example 18 is the method of Example 17, wherein the macroporous polymer resin consists of the Dowex™ Optipore™ L493.

Example 19 is a system for producing an ethanol product. The system includes a vessel fluidly connected to a liquid feed stream. The liquid feed stream includes ethanol at a concentration greater than 30% by volume. The vessel contains an adsorbent polymer resin. The adsorbent polymer resin includes a polystyrene divinylbenzene copolymer. A surface of the adsorbent polymer resin is hydrophobic.

Example 20 is the system of Example 19, wherein the adsorbent polymer resin is a macroporous polymer resin including at least one of: Dowex™ Optipore™ L493 adsorbent resin and Amberlite™ FPX66 adsorbent resin.

Example 21 is a method for producing a purified ethanol product. The method includes obtaining a vapor phase distillate including ethanol at a concentration greater than 30% by volume, contacting the vapor phase distillate with an adsorbent polymer resin, and condensing the vapor phase distillate to produce the purified ethanol product. The adsorbent polymer resin includes a polystyrene divinylbenzene copolymer. A surface of the adsorbent polymer resin is hydrophobic.

Example 22 is the method of Example 21, wherein contacting the vapor phase distillate with the adsorbent polymer resin includes flowing the vapor phase distillate through a vessel containing the adsorbent polymer resin.

Example 23 is the method of Example 21, further including regenerating the adsorbent polymer resin.

Example 24 is the method of Example 23, wherein regenerating the adsorbent polymer resin includes contacting the adsorbent polymer resin with at least one of: steam and sodium hydroxide.

Example 25 is the method of any of Examples 21-24, wherein the concentration of the ethanol in the vapor phase distillate is between 30% and 95%.

Example 26 is the method of Example 25, wherein concentration of the ethanol in the vapor phase distillate is between 80% and 96%.

Example 27 is the method of any of Examples 21-26, wherein the adsorbent polymer resin is a macroporous polymer resin.

Example 28 is the method of Example 27, wherein the macroporous polymer resin includes of: Dowex™ Optipore™ V493 adsorbent resin.

Example 29 is the method of Example 28, wherein the macroporous polymer resin consists of the Dowex™ Optipore™ V493 adsorbent resin.

Example 30 is a method for producing an ethanol product. The method includes obtaining a vapor phase distillate including ethanol at a concentration greater than 30% by volume, contacting the vapor phase distillate with a plurality of adsorbent materials, and condensing the vapor phase distillate to produce the purified ethanol product. The plurality of adsorbent materials includes an activated carbon and a polymer resin. The polymer resin includes a polystyrene divinylbenzene copolymer. A surface of the polymer resin is hydrophobic.

Example 31 is the method of Example 30, wherein contacting the vapor phase distillate with the plurality of adsorbent materials includes flowing the vapor phase distillate through at least one vessel containing at least one of the plurality of adsorbent materials.

Example 32 is the method of either of Examples 30 or 31, wherein contacting the vapor phase distillate with the plurality of adsorbent materials includes contacting the vapor phase distillate with the activated carbon before contacting the vapor phase distillate with the polymer resin.

Example 33 is the method of any of Examples 30-32, wherein the plurality of adsorbent materials consists of the activated carbon and the polymer resin.

Example 34 is the method of any of Examples 30-33, wherein the polymer resin is between 1 wt. % and 99 wt. % of the plurality of adsorbent materials.

Example 35 is the method of any of Examples 30-34, wherein the concentration of the ethanol in the vapor phase distillate is between 30% and 95%.

Example 36 is the method of any of Examples 30-35, wherein the polymer resin is a macroporous polymer resin.

Example 37 is the method of Example 36, wherein the macroporous polymer resin includes Dowex™ Optipore™ V493 adsorbent resin.

Example 38 is the method of Example 37, wherein the macroporous polymer resin consists of the Dowex™ Optipore™ V493.

Example 39 is a system for producing an ethanol product. The system includes a vessel fluidly connected to a vapor phase feed stream including ethanol at a concentration greater than 30% by volume. The vessel contains an adsorbent polymer resin. The adsorbent polymer resin includes a polystyrene divinylbenzene copolymer. A surface of the adsorbent polymer resin is hydrophobic.

Example 40 is the system of Example 39, wherein the adsorbent polymer resin is a macroporous polymer resin including Dowex™ Optipore™ V493 adsorbent resin.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 is a schematic illustration of a system for producing an ethanol product, according to some embodiments of this disclosure.

FIG. 2 is a schematic illustration of another system for producing an ethanol product, according to some embodiments of this disclosure.

FIG. 3 is a schematic illustration of another system for producing an ethanol product, according to some embodiments of this disclosure.

FIG. 4 is a schematic illustration of a system for producing an ethanol product, according to some embodiments of this disclosure.

FIG. 5 is a schematic illustration of another system for producing an ethanol product, according to some embodiments of this disclosure.

FIG. 6 is a schematic illustration of another system for producing an ethanol product, according to some embodiments of this disclosure.

FIG. 7 is a graph illustrating a relative concentration of 2-heptanone as a function of passes and dwell times in contact with an adsorbent polymer resin, according to some embodiments of this disclosure.

FIG. 8 is a graph illustrating a relative concentration of 2-octanone as a function of passes and dwell times in contact with an adsorbent polymer resin, according to some embodiments of this disclosure.

FIG. 9 is a graph illustrating a relative concentration of furfural as a function of passes and dwell times in contact with an adsorbent polymer resin, according to some embodiments of this disclosure.

FIG. 10 is a graph illustrating a relative concentration of benzaldehyde as a function of passes and dwell times in contact with an adsorbent polymer resin, according to some embodiments of this disclosure.

FIG. 11 is a graph illustrating a relative concentration of acetophenone as a function of passes and dwell times in contact with an adsorbent polymer resin, according to some embodiments of this disclosure.

### DETAILED DESCRIPTION

Purification of ethanol products from fermentation products can be accomplished to varying degrees with a number of methods known in the art. However, in many of these methods, a significant portion of the ethanol present in the ethanol product prior to purification is removed in the purification process. The resulting purified ethanol product may not be as purified as desired, and/or may require a costly addition of ethanol to make up for the ethanol lost in the purification process to provide an ethanol product having a desired concentration of ethanol.

Embodiments of the present disclosure employ an adsorbent polymer resin including a polystyrene divinylbenzene polymer to purify or modify an ethanol product. The ethanol product can have an ethanol concentration greater than 30% by volume. The adsorbent polymer resin is substantially non-functionalized and is hydrophobic. It has been found that the use of the adsorbent polymer resin in accordance with embodiments of this disclosure can remove organic compounds that may compromise or otherwise provide an undesired color, flavor, and/or aroma of the ethanol product, including 2-heptanone, 2-octanone, and furfural. The ethanol concentration is not expected to change significantly in the purification process. Without wishing to be bound by any theory, it is believed that the hydrophobic nature of the adsorbent polymer resin resists adsorption of ethanol, which is a relatively polar compound, while adsorbing non-polymer organic compounds, such as 2-heptanone, 2-octanone, and furfural.

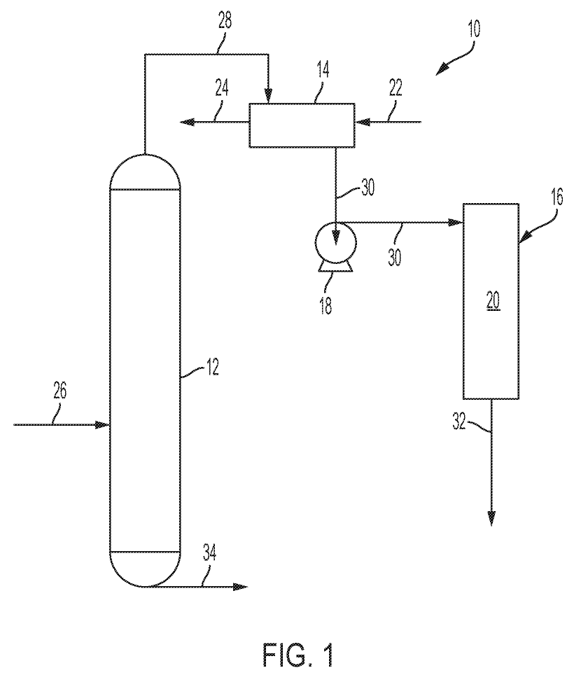

FIG. 1 is a schematic illustration of a system for producing an ethanol product, according to some embodiments of this disclosure. FIG. 1 shows system 10 including a distillation column 12, a condenser 14, and vessel 16, and a pump 18. The vessel 16 includes a polymer resin 20. The distillation column 12 can be, for example, a multi-tray distillation column or a packed distillation column, as are known in the art. The condenser 14 can include a cooling fluid input 22 and a cooling fluid output 24. The vessel 16 can be, for example, a column, a vat, or other suitable vessel. The system 10 further includes a fermentation feed stream 26, a distillate vapor stream 28, a condensed distillate stream 30, and a purified product stream 32. The purified product stream 32 can be an ethanol product.

The polymer resin 20 can be an adsorbent polymer resin including a polystyrene divinylbenzene polymer. In some embodiments, the polymer resin 20 can be a styrenic polymer cross-linked with divinylbenzene. In some embodiments, the polymer resin 20 can be in the form of a plurality of spherical beads forming a resin bed within the vessel 16. The cross-linked structure can provide the spherical beads of the polymer resin 20 with high crush strength to maintain open fluid paths through the resin bed. In some embodiments, the polymer resin 20 can be in the form of a fiber mesh within the vessel 16.

The polymer resin 20 can be a hydrophobic resin. In some embodiments, the polymer resin 20 can be substantially non-functionalized. That is, a surface of the polymer resin 20 is substantially free of functional groups grafted, or covalently bonded, to surface of the polymer resin 20. As noted above, it is believed that the hydrophobic nature of the surface of the polymer resin 20 reduces the adsorption of ethanol compared with polymer resins that are hydrophilic, or polymer resins that are hydrophobic but have hydrophilic functional groups grafted to their surfaces.

In some embodiments, the polymer resin 20 can be macroporous. A macroporous resin, also referred to as a macroreticular resin, can have a porous or multi-channel structure to provide a large surface area per gram of resin. For example, in some embodiments, the polymer resin 20 can have a Brunauer, Emmett and Teller (BET) surface area greater than 700 m²/g. In some embodiments, the polymer resin 20 can have a BET surface area greater than 1,100 m²/g.

In some embodiments, the polymer resin 20 can include Dowex™ Optipore™ L493 adsorbent resin, from the Dow Chemical Company, Midland, Mich. In some embodiments, the polymer resin 20 can consist essentially of Dowex™ Optipore™ L493 adsorbent resin. In some embodiments, the polymer resin 20 can consist of Dowex™ Optipore™ L493 adsorbent resin. In some embodiments, the polymer resin 20 can include Amberlite™ FPX66 adsorbent resin, also from the Dow Chemical Company, Midland, Mich. In some embodiments, the polymer resin 20 can consist essentially of Amberlite™ FPX66 adsorbent resin. In some embodiments, the polymer resin 20 can consist of Amberlite™ FPX66 adsorbent resin. In some embodiments, the polymer resin 20 can include a combination of any of the previously mentioned adsorbent resins.

In some embodiments, prior to use, the polymer resin 20 can be rinsed with a solvent, such as methanol. Such a pre-use treatment may remove residual compounds involved in the manufacture of the polymer resin 20.

In some embodiments, the cooling fluid input 22 can provide a flow of cooling fluid to the condenser 14 and cooling fluid output 24 can provide a flow of the cooling fluid from the condenser 14. Together, the cooling fluid flow provided by the cooling fluid input 22 and the cooling fluid output 24 can maintain portions of the condenser 14 at a condensation temperature. In some embodiments, the cooling fluid can include water.

The fermentation feed stream 26 can provide a stream of fermentation products. The fermentation feed stream 26 can be produced from the fermentation of plant products by a yeast. The plant products can include, for example, fruits, tubers, grains, honey, and/or sugar cane. Such fermentation products can have an ethanol concentration less than about 19% by volume, as concentrations exceeding 19% can be toxic to yeasts used in creating the fermentation feed stream 26.

Referring to FIG. 1, the fermentation feed stream 26 is fluidly connected to the distillation column 12. The distillate vapor stream 28 fluidly connects the condenser 14 to the distillation column 12. The condensed distillate stream 30 fluidly connects the vessel 16 to the condenser 14. The purified product stream 32 fluidly connects to the vessel 16. In some embodiments, the pump 18 is disposed along the condensed distillate stream 30.

In use, fermentation products can flow into the distillation column 12 though the fermentation feed stream 26. The distillation column 12 can separate the fermentation products into the distillate vapor stream 28 including ethanol at a concentration greater than 30% by volume, and a bottoms liquid stream 34 including very little ethanol. The distillate vapor stream 28 can flow to the condenser 14 where the low temperature surfaces of the condenser 14 condense the distillate vapor stream 28 to form the condensed distillate stream 30.

The condensed distillate stream 30 can flow into the vessel 16, either by gravity or pumped by the pump 18. As noted above, the condensed distillate stream 30 may include organic compounds which may compromise the color, flavor, and/or aroma of ethanol product. Within the vessel 16, the condensed distillate stream 30 can contact the polymer resin 20. The organic compounds can adsorb onto the hydrophobic surface of the polymer resin 20, removing it from the condensed distillate stream 30, resulting in the purified product stream 32 flowing from the vessel 16. The ethanol concentration does not decrease significantly between the condensed distillate stream 30 flowing into the vessel 16 and the purified product stream 32 flowing out of the vessel 16.

The condensed distillate stream 30 includes ethanol at a concentration greater than 30%. In some embodiments, the condensed distillate stream 30 includes ethanol at a concentration between 30% and 95%. In some embodiments, the condensed distillate stream 30 includes an ethanol concentration as low as 35%, 40%, 45%, 50%, 55%, or 60%, or as high as 65%, 70%, 75%, 80%, 85%, 90%, or 95%, or at any concentration between any two of the preceding concentrations. For example, in some embodiments, the ethanol concentration can range from 35% to 95%, 40% to 90%, 45%, to 85%, 50% to 80%, 55% to 75%, 60% to 70%, 65% to 95%, 80% to 95%, or 90% to 95%. All percentages of ethanol are by volume.

The polymer resin 20 has a finite capacity to adsorb the organic compounds. Thus, periodically, the polymer resin 20 must be regenerated to be reused. For example, the polymer resin 20 can be taken off-line from the system 10 and the polymer resin 20 can be contacted with a material able to desorb the adsorbed organic compounds. In some embodiments, the material can include steam. In some embodiments, the material can include a sodium hydroxide solution. The cross-linked polystyrene divinylbenzene polymer of the polymer resin 20 can be resistant to chemical attack from strong bases as well as strong acids.

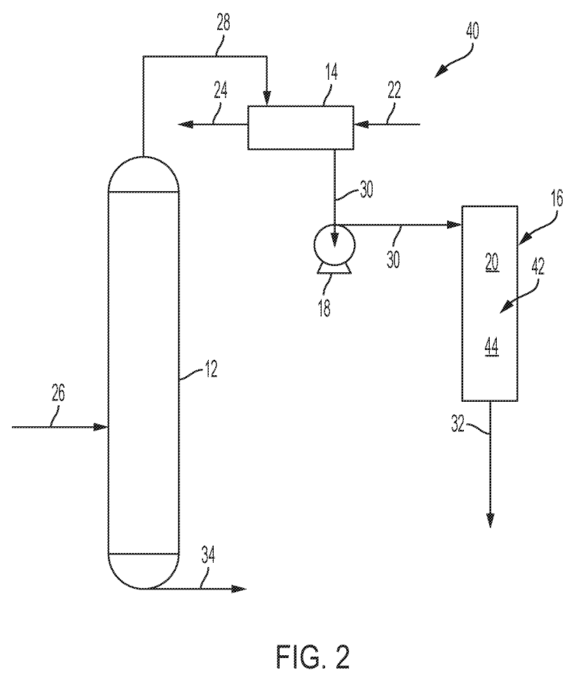

FIG. 2 is a schematic illustration of another system for producing an ethanol product, according to some embodiments of this disclosure. FIG. 2 illustrates a system 40. The system 40 is substantially similar to the system 10 described above in reference to FIG. 1, except that the vessel 16 further includes a plurality of adsorbent materials 42. The plurality of adsorbent materials 42 includes the polymer resin 20, as described above in reference to FIG. 1, and an activated carbon 44. In some embodiments, the activated carbon 44 can include an uncoated synthetic charcoal or an uncoated organic charcoal. In some embodiments, the activated carbon 44 can included uncoated coconut shell granule charcoal.

In some embodiments, the weight percentage of the polymer resin 20 included in the plurality of adsorbent materials 42 can be as little as 1 weight percent (wt. %), 5 wt. %, 10 wt. %, 15 wt. %, 20 wt. %, 25 wt. %, 30 wt. %, 35 wt. %, 40 wt. %, 45 wt. %, or 50 wt. %, or as much as 55 wt. %, 60 wt. %, 65 wt. %, 70 wt. %, 75 wt. %, 80 wt. %, 85 wt. %, 90 wt. %, 95 wt. %, or 99 wt. %, or any amount between any of the preceding amounts. For example, in some embodiments, the weight percentage of the polymer resin 20 included in the plurality of adsorbent materials 42 can range from 1 wt. % to 99 wt. %, 5 wt. % to 95 wt. %, 10 wt. % to 90 wt. %, 15 wt. % to 85 wt. %, 20 wt. % to 80 wt. %, 25 wt. % to 75 wt. %, 30 wt. % to 70 wt. %, 35 wt. % to 65 wt. %, 40 wt. % to 60 wt. %, 45 wt. % to 55 wt. %, 5 wt. % to 40 wt. %, or 10 wt. % to 30 wt. %. In some embodiments, the plurality of adsorbent materials 42 can consist essentially of the polymer resin 20 and the activated carbon 44. In some embodiments, the plurality of adsorbent materials 42 can consist of the polymer resin 20 and the activated carbon 44.

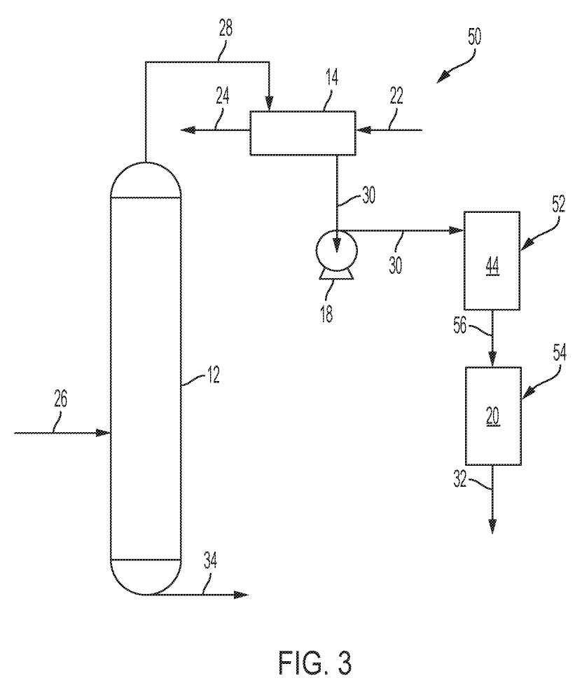

FIG. 3 is a schematic illustration of another system for producing an ethanol product, according to some embodiments of this disclosure. FIG. 3 illustrates a system 50. The system 50 is substantially similar to the system 10 described above in reference to FIG. 1, except that the vessel 16 is replaced by a first vessel 52, a second vessel 54, and an intermediate purified product stream 56. The first vessel 52 can include the activated carbon 44 and the second vessel 54 can include the polymer resin 20, each as described above in reference to FIG. 1. The first vessel 52 and the second vessel 54 can each be, for example, a column, a vat, or other suitable vessel. In some embodiments, the first vessel 52 and the second vessel 54 can be the same type of vessel.

Referring to FIG. 3, the fermentation feed stream 26 is fluidly connected to the distillation column 12. The distillate vapor stream 28 fluidly connects the condenser 14 to the distillation column 12. The condensed distillate stream 30 fluidly connects the first vessel 52 to the condenser 14. The intermediate purified product stream 56 fluidly connects the first vessel 52 to the second vessel 54. The purified product stream 32 fluidly connects to the second vessel 54. In some embodiments, the pump 18 is disposed along the condensed distillate stream 30.

In use, the condensed distillate stream 30 can flow into the first vessel 52, either by gravity or pumped by the pump 18. Within the first vessel 52, the condensed distillate stream 30 can contact the activated carbon 44. Some organic compounds can adsorb onto the activated carbon 44, resulting in the intermediate purified product stream 56. The intermediate purified product stream 56 can flow into the second vessel 54. Within the second vessel 54, the intermediate purified product stream 56 can contact the polymer resin 20. Some remaining organic compounds can adsorb onto the hydrophobic surface of the polymer resin 20, removing them from the intermediate purified product stream 56, resulting in the purified product stream 32 flowing from the second vessel 54. The ethanol concentration does not decrease significantly between the intermediate purified product stream 56 flowing into the second vessel 54 and the purified product stream 32 flowing out of the second vessel 54.

In the embodiment, shown in FIG. 3, the condensed distillate stream 30 is brought into contact with the activated carbon 44 before it is brought into contact with the polymer resin 20. However, in other embodiments, the first vessel 52 can include the polymer resin 20, and the second vessel 54 can include the activated carbon 44, such that the condensed distillate stream 30 is brought into contact with the polymer resin 20 before it is brought into contact with the activated carbon 44.

In the embodiments described above, the distillation column 12 is a single column. However, it is understood that the disclosure includes embodiments including multiple distillation columns 12. Further, the embodiments describe above are described as continuous flow systems. However, it is understood that the disclosure includes batch embodiments as well.

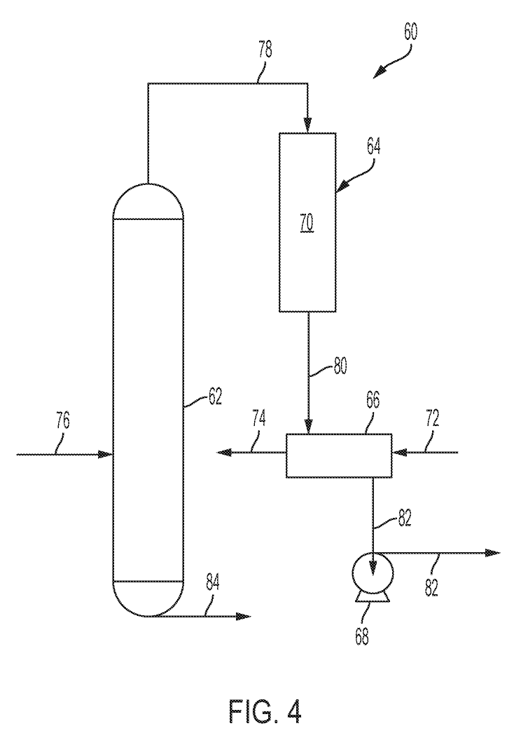

FIG. 4 is a schematic illustration of another system for producing an ethanol product, according to some embodiments of this disclosure. FIG. 4 shows system 60 including a distillation column 62, a vessel 64, a condenser 66, and a pump 88. The vessel 64 includes a polymer resin 70. The distillation column 62 can be, for example, a multi-tray distillation column or a packed distillation column, as are known in the art. The vessel 64 can be, for example, a column, a vat, or other suitable vessel. The condenser 66 can include a cooling fluid input 72 and a cooling fluid output 74. The system 60 further includes a fermentation feed stream 76, a distillate vapor stream 78, a purified vapor stream 80, and a purified condensed distillate stream 82. The purified condensed distillate stream 82 can be an ethanol product.

The polymer resin 70 can be an adsorbent polymer resin including a polystyrene divinylbenzene polymer. In some embodiments, the polymer resin 70 can be a styrenic polymer cross-linked with divinylbenzene. In some embodiments, the polymer resin 70 can be in the form of a plurality of spherical beads forming a resin bed within the vessel 64. The cross-linked structure can provide the spherical beads of the polymer resin 70 with high crush strength to maintain open fluid paths through the resin bed. In some embodiments, the polymer resin 70 can be in the form of a fiber mesh within the vessel 64.

The polymer resin 70 can be a hydrophobic resin. In some embodiments, the polymer resin 70 can be substantially non-functionalized. That is, a surface of the polymer resin 70 is substantially free of functional groups grafted, or covalently bonded, to surface of the polymer resin 70. As noted above, it is believed that the hydrophobic nature of the surface of the polymer resin 70 reduces the adsorption of ethanol compared with polymer resins that are hydrophilic, or polymer resins that are hydrophobic but have hydrophilic functional groups grafted to their surfaces.

In some embodiments, the polymer resin 70 can be macroporous. A macroporous resin, also referred to as a macroreticular resin, can have a porous or multi-channel structure to provide a large surface area per gram of resin. For example, in some embodiments, the polymer resin 70 can have a Brunauer, Emmett and Teller (BET) surface area greater than 700 m²/g. In some embodiments, the polymer resin 70 can have a BET surface area greater than 1,100 m²/g.

In some embodiments, the polymer resin 70 can include Dowex™ Optipore™ V493 adsorbent resin, from the Dow Chemical Company, Midland, Mich. In some embodiments, the polymer resin 70 can consist essentially of Dowex™ Optipore™ V493 adsorbent resin. In some embodiments, the polymer resin 70 can consist of Dowex™ Optipore™ V493 adsorbent resin.

In some embodiments, prior to use, the polymer resin 70 can be rinsed with a solvent, such as methanol. Such a pre-use treatment may remove residual compounds involved in the manufacture of the polymer resin 70.

In some embodiments, the cooling fluid input 72 can provide a flow of cooling fluid to the condenser 66 and cooling fluid output 74 can provide a flow of the cooling fluid from the condenser 66. Together, the cooling fluid flow provided by the cooling fluid input 72 and the cooling fluid output 74 can maintain portions of the condenser 66 at a condensation temperature. In some embodiments, the cooling fluid can include water.

The fermentation feed stream 76 can provide a stream of fermentation products. The fermentation feed stream 76 can be produced from the fermentation of plant products by a yeast. The plant products can include, for example, fruits, tubers, grains, honey, and/or sugar cane. Such fermentation products can have an ethanol concentration less than about 19% by volume, as concentrations exceeding 19% can be toxic to yeasts used in creating the fermentation feed stream 76.

Referring to FIG. 4, the fermentation feed stream 76 is fluidly connected to the distillation column 62. The distillate vapor stream 78 fluidly connects the vessel 64 to the distillation column 62. The purified vapor stream 80 fluidly connects the vessel 64 to the condenser 66. The purified condensed distillate stream 82 fluidly connects to the condenser 66. In some embodiments, the pump 68 is disposed along the purified condensed distillate stream 82.

In use, fermentation products can flow into the distillation column 62 though the fermentation feed stream 76. The distillation column 62 can separate the fermentation products into the distillate vapor stream 78 including ethanol at a concentration greater than 30% by volume, and a bottoms liquid stream 84 including very little ethanol. The distillate vapor stream 78 includes ethanol at a concentration greater than 30%. In some embodiments, the distillate vapor stream 78 includes ethanol at a concentration between 30% and 95%. In some embodiments, the distillate vapor stream 78 includes an ethanol concentration as low as 35%, 40%, 45%, 50%, 55%, or 60%, or as high as 65%, 70%, 75%, 80%, 85%, 90%, or 95%, or at any concentration between any two of the preceding concentrations. For example, in some embodiments, the ethanol concentration can range from 35% to 95%, 40% to 90%, 45%, to 85%, 50% to 80%, 55% to 75%, 60% to 70%, 65% to 95%, 80% to 95%, or 90% to 95%. All percentages of ethanol are by volume.

The distillate vapor stream 78 can flow into the vessel 64. The distillate vapor stream 78 may include organic compounds which may compromise the color, flavor, and/or aroma of ethanol product. Within the vessel 64, the distillate vapor stream 78 can contact the polymer resin 70. The organic compounds can adsorb onto the hydrophobic surface of the polymer resin 70, removing it from the distillate vapor stream 78, resulting in the purified vapor stream 80 flowing from the vessel 64. The ethanol concentration does not decrease significantly between the distillate vapor stream 78 flowing into the vessel 64 and the purified vapor stream 80 flowing out of the vessel 64.

The purified vapor stream 80 can flow into the condenser 66 where the low temperature surfaces of the condenser 66 condense the purified vapor stream 80 to form the purified condensed distillate stream 82. The purified condensed distillate stream 82 can flow out of the condenser 66, either by gravity or pumped by the pump 68.

The polymer resin 70 has a finite capacity to adsorb the organic compounds. Thus, periodically, the polymer resin 70 must be regenerated to be reused. For example, the polymer resin 70 can be taken off-line from the system 60 and the polymer resin 70 can be contacted with a material able to desorb the adsorbed organic compounds. In some embodiments, the material can include steam. In some embodiments, the material can include a sodium hydroxide solution. The cross-linked polystyrene divinylbenzene polymer of the polymer resin 70 can be resistant to chemical attack from strong bases as well as strong acids.

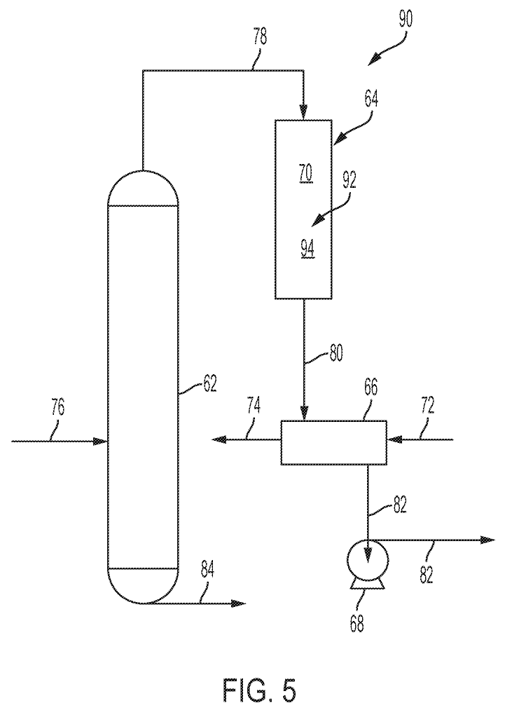

FIG. 5 is a schematic illustration of another system for producing an ethanol product, according to some embodiments of this disclosure. FIG. 5 illustrates a system 90. The system 90 is substantially similar to the system 60 described above in reference to FIG. 4, except that the vessel 64 further includes a plurality of adsorbent materials 92. The plurality of adsorbent materials 92 includes the polymer resin 70, as described above in reference to FIG. 5, and an activated carbon 94. In some embodiments, the activated carbon 94 can include an uncoated synthetic charcoal or an uncoated organic charcoal. In some embodiments, the activated carbon 94 can included uncoated coconut shell granule charcoal.

In some embodiments, the weight percentage of the polymer resin 70 included in the plurality of adsorbent materials 92 can be as little as 1 weight percent (wt. %), 5 wt. %, 10 wt. %, 15 wt. %, 20 wt. %, 25 wt. %, 30 wt. %, 35 wt. %, 40 wt. %, 45 wt. %, or 50 wt. %, or as much as 55 wt. %, 60 wt. %, 65 wt. %, 70 wt. %, 75 wt. %, 80 wt. %, 85 wt. %, 90 wt. %, 95 wt. %, or 99 wt. %, or any amount between any of the preceding amounts. For example, in some embodiments, the weight percentage of the polymer resin 70 included in the plurality of adsorbent materials 92 can range from 1 wt. % to 99 wt. %, 5 wt. % to 95 wt. %, 10 wt. % to 90 wt. %, 15 wt. % to 85 wt. %, 20 wt. % to 80 wt. %, 25 wt. % to 75 wt. %, 30 wt. % to 70 wt. %, 35 wt. % to 65 wt. %, 40 wt. % to 60 wt. %, 45 wt. % to 55 wt. %, 5 wt. % to 40 wt. %, or 10 wt. % to 30 wt. %. In some embodiments, the plurality of adsorbent materials 92 can consist essentially of the polymer resin 70 and the activated carbon 94. In some embodiments, the plurality of adsorbent materials 92 can consist of the polymer resin 70 and the activated carbon 94.

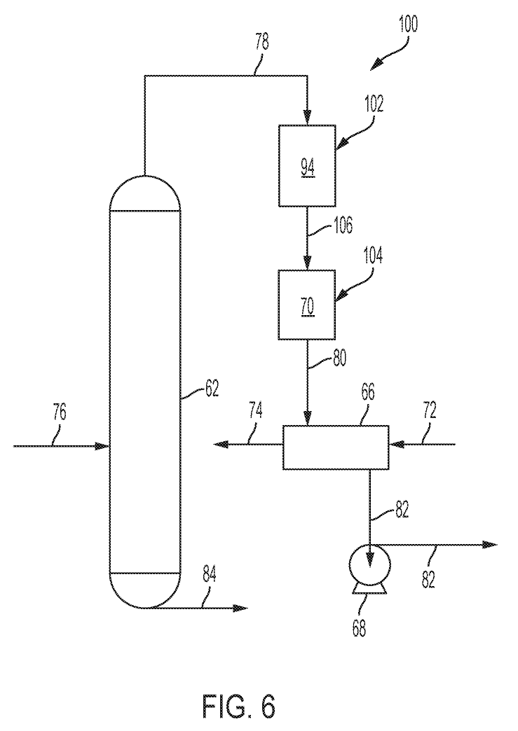

FIG. 6 is a schematic illustration of another system for producing an ethanol product, according to some embodiments of this disclosure. FIG. 6 illustrates a system 100. The system 100 is substantially similar to the system 60 described above in reference to FIG. 4, except that the vessel 64 is replaced by a first vessel 102, a second vessel 104, and an intermediate purified vapor stream 106. The first vessel 102 can include the activated carbon 94 and the second vessel 104 can include the polymer resin 70, each as described above in reference to FIG. 4. The first vessel 102 and the second vessel 104 can each be, for example, a column, a vat, or other suitable vessel. In some embodiments, the first vessel 102 and the second vessel 104 can be the same type of vessel.

Referring to FIG. 6, the fermentation feed stream 76 is fluidly connected to the distillation column 62. The distillate vapor stream 78 fluidly connects the first vessel 102 to the distillation column 62. The intermediate purified vapor stream 106 fluidly connects the first vessel 102 to the second vessel 104. The purified vapor stream 80 fluidly connects the second vessel 104 to the condenser 66. The purified condensed distillate stream 82 fluidly connects to the condenser 66. In some embodiments, the pump 68 is disposed along the purified condensed distillate stream 82.

In use, the distillate vapor stream 78 can flow into the first vessel 102. Within the first vessel 102, the distillate vapor stream 78 can contact the activated carbon 94. Some organic compounds can adsorb onto the activated carbon 94, resulting in the intermediate purified vapor stream 106. The intermediate purified vapor stream 106 can flow into the second vessel 104. Within the second vessel 104, the intermediate purified vapor stream 106 can contact the polymer resin 70. Some remaining organic compounds can adsorb onto the hydrophobic surface of the polymer resin 70, removing them from the intermediate purified vapor stream 106, resulting in the purified vapor stream 80 flowing from the second vessel 104 and into the condenser 66. The ethanol concentration does not decrease significantly between the intermediate purified vapor stream 106 flowing into the second vessel 104 and the purified vapor stream 80 flowing into the condenser 66.

In the embodiment, shown in FIG. 6, the distillate vapor stream 78 is brought into contact with the activated carbon 94 before it is brought into contact with the polymer resin 70. However, in other embodiments, the first vessel 102 can include the polymer resin 70, and the second vessel 104 can include the activated carbon 94, such that the distillate vapor stream 78 is brought into contact with the polymer resin 70 before it is brought into contact with the activated carbon 94.

In the embodiments described above, the distillation column 62 is a single column. However, it is understood that the disclosure includes embodiments including multiple distillation columns 62. Further, the embodiments describe above are described as continuous flow systems. However, it is understood that the disclosure includes batch embodiments as well.

A purified ethanol product produced as described above may be used as a beverage, such as a vodka, for example. A purified ethanol product produced as described above may be used as an external medical product, such as a disinfectant, for example. A purified ethanol product produced as described above may be used as in an internal medical product.

### EXAMPLES

The present invention is more particularly described in the following examples that are intended as illustrations only, since numerous modifications and variations within the scope of the present invention will be apparent to those of skill in the art.

### Example—Ketone and Aldehyde Concentration in Vodka Samples

The concentrations of five organic compounds in samples of a test vodka were measured before and after purifying according to some embodiments of this disclosure. The test vodka included a commercially obtained vodka combined with vodka from the heads and tails portions of a batch vodka production process. The test vodka had an ethanol concentration of in excess of 90% by volume. Six samples of the test vodka were purified. For Sample 1, 250 ml of the test vodka was passed without any dwell time over 100 g of unused Dowex™ Optipore™ L493 adsorbent resin. For Sample 2, another 250 ml of the test vodka was brought into contact for a dwell time of 30 seconds with the same 100 g of resin used to purify Sample 1. For Sample 3, another 250 ml of the test vodka was passed five times (without any dwell time) over the same 100 g of resin used to purify Samples 1 and 2. For sample 4, another 250 ml of the test vodka was passed ten times (without any dwell time) over the same 100 g of resin used to purify samples 1, 2, and 3. For Sample 5, 50 ml of the test vodka was passed five times (without any dwell time) over 100 g of unused Dowex™ Optipore™ L493 adsorbent resin. For Sample 6, 170 ml of the test vodka was passed ten times (without any dwell time) over the same 100 g of resin used to purify Sample 5.

Each of the Samples 1-5 were subjectively evaluated for taste and aroma compared to the original test vodka which was not purified according to embodiments of this disclosure. Each of the Samples 1-5 were found to exhibit a noticeable improvement in taste and aroma compared to the original test vodka.

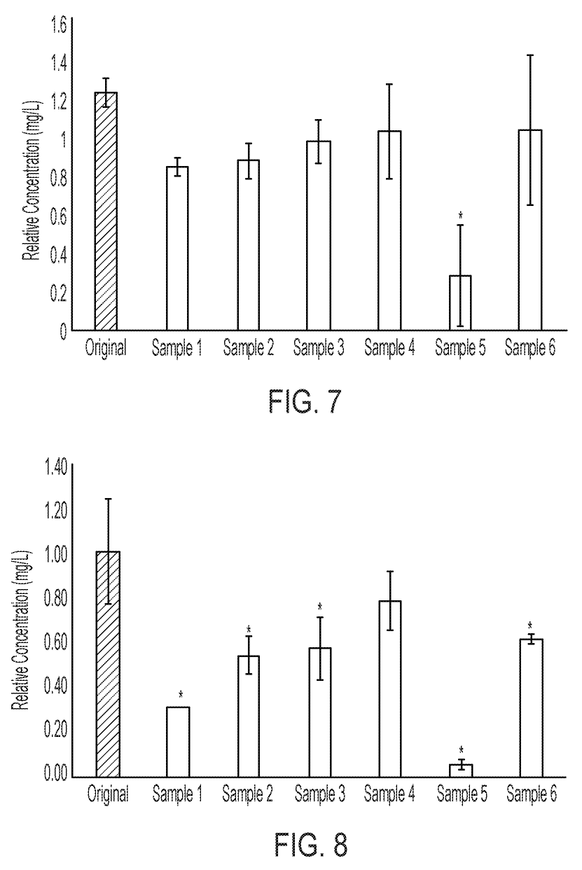

A control sample of the original vodka not purified according to embodiments of this disclosure and each of the Samples 1-5 were quantified for 2-heptanone, 2-octanone, furfural, benzaldehyde, and acetophenone. Each of the Samples 1-5 and the original vodka were individually quantified by Dynamic Headspace and Gas Chromatography/Mass Spectrometry by placing 2.5 ml in a 20 ml headspace vial with 2.5 ml of water and spiked with 1.8 μg of 2-methyl-3-heptanone to be used as internal standard. The six vials were placed in an incubator at 50° C. and purged with nitrogen at a flow rate of 50 ml/min for a total volume of 800 ml. A trap (Tenax TA) was then dry purged at a rate of 100 ml/min and transferred to a thermal desorption unit. Volatile compounds were desorbed from the trap, cryofocused at −50° C., and injected onto the column in solvent vent mode. The volatile compounds were separated using an Agilent 7890B GC (Agilent Technologies, Santa Clara, Calif.) equipped with a DB-Wax column (60 m×0.250 mm×0.25 μm, Agilent Technologies) and detected with a 7010B Triple Quadrupole Mass Spectrometer (QqQ-MS) (Agilent Technologies). The initial oven temperature was 40° C., ramped at 4° C./min to 190° C. with a 1 min hold, and then ramped at 120° C./min to 230° C. with a 5 min hold. Helium was used as a carrier gas at a constant flow rate of 1.6 ml/min. The mass scanning range waw 29-400 m/z. The mass spectra were obtained at 70 eV in the electron ionization (EI) mode. Relative concentrations of the five organic compounds were calculated using internal standard response factor. The results are show in FIGS. 7-11.

FIG. 7 is a graph illustrating a relative concentration of 2-heptanone for the original vodka and each of the Samples 1-5. As shown in FIG. 7, the relative concentration of 2-heptanone was reduced in each of the Samples 1-5.

FIG. 8 is a graph illustrating a relative concentration of 2-octanone for the original vodka and each of the Samples 1-5. As shown in FIG. 8, the relative concentration of 2-octanone was reduced in each of the Samples 1-5.

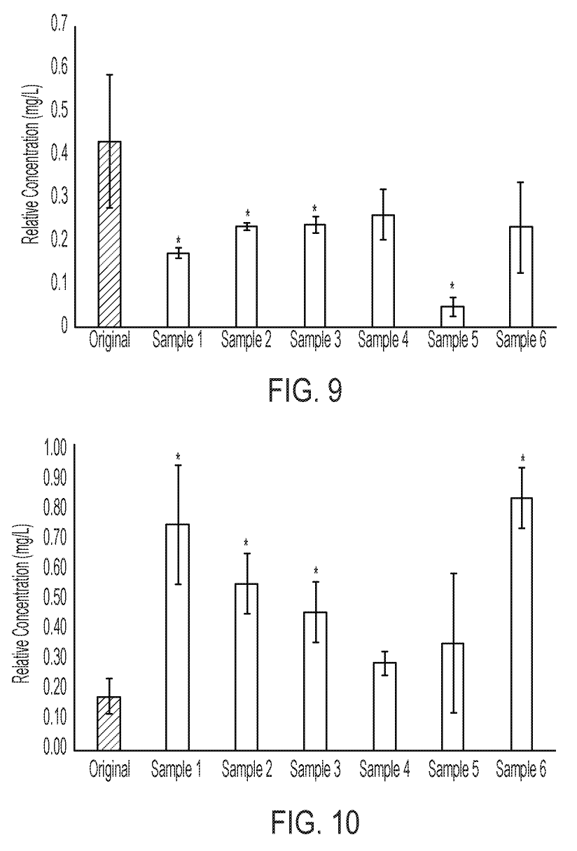

FIG. 9 is a graph illustrating a relative concentration of furfural for the original vodka and each of the Samples 1-5. As shown in FIG. 9, the relative concentration of furfural was reduced in each of the Samples 1-5.

FIG. 10 is a graph illustrating a relative concentration of benzaldehyde for the original vodka and each of the Samples 1-5. As shown in FIG. 10, the relative concentration of benzaldehyde was not reduced in any of the Samples 1-5.

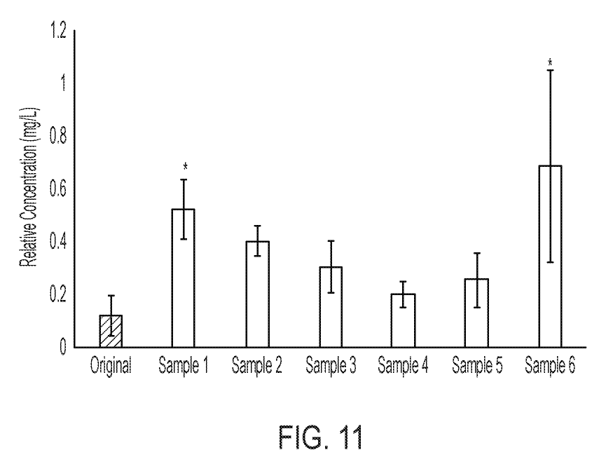

FIG. 11 is a graph illustrating a relative concentration of acetophenone for the original vodka and each of the Samples 1-5. As shown in FIG. 11, the relative concentration of acetophenone was not reduced in any of the Samples 1-5.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above described features.

What is claimed is:

1. A method for producing an ethanol product, the method comprising:
obtaining a condensed distillate including ethanol at a concentration greater than 30% by volume; and
contacting the condensed distillate with at least one adsorbent material including an adsorbent polymer resin, the adsorbent polymer resin including a polystyrene divinylbenzene copolymer, wherein a surface of the adsorbent polymer resin is hydrophobic.

2. The method of claim 1, wherein the concentration of the ethanol in the condensed distillate is between 30% and 95%.

3. The method of claim 1, wherein the adsorbent polymer resin is a macroporous polymer resin.

4. The method of claim 3, wherein the macroporous polymer resin is at least one selected from the group of Dowex™ Optipore™ L493 adsorbent resin and Amberlite™ FPX66 adsorbent resin.

5. The method of claim 1, wherein the at least one adsorbent material is a plurality of adsorbent materials, the plurality of adsorbent materials further including an activated carbon.

6. The method of claim 5, wherein contacting the condensed distillate with the plurality of adsorbent materials includes flowing the condensed distillate through a vessel containing the plurality of adsorbent materials.

7. The method of claim 5, wherein contacting the condensed distillate with the plurality of adsorbent materials includes contacting the condensed distillate with the activated carbon before contacting the condensed distillate with the polymer resin.

8. The method of claim 5, wherein contacting the condensed distillate with the plurality of adsorbent materials includes contacting the condensed distillate with the polymer resin before contacting the condensed distillate with the activated carbon.

9. A method for producing a purified ethanol product, the method comprising:
obtaining a vapor phase distillate including ethanol at a concentration greater than 30% by volume;
contacting the vapor phase distillate with at least one adsorbent material including an adsorbent polymer resin, the adsorbent polymer resin including a polystyrene divinylbenzene copolymer, wherein a surface of the adsorbent polymer resin is hydrophobic; and
condensing the vapor phase distillate to produce the purified ethanol product.

10. The method of claim 9, wherein the concentration of the ethanol in the vapor phase distillate is between 30% and 95%.

11. The method of claim 9, wherein the adsorbent polymer resin is a macroporous polymer resin.

12. The method of claim 11, wherein the macroporous polymer resin includes Dowex™ Optipore™ V493 adsorbent resin.

13. The method of claim 9, wherein the at least one adsorbent material is a plurality of adsorbent materials, the plurality of adsorbent materials further including an activated carbon.

14. The method of claim 9, wherein contacting the vapor phase distillate with the plurality of adsorbent materials includes flowing the vapor phase distillate through a vessel containing the plurality of adsorbent materials.

15. The method of claim 9, wherein contacting the vapor phase distillate with the plurality of adsorbent materials includes contacting the vapor phase distillate with the activated carbon before contacting the vapor phase distillate with the polymer resin.

16. The method of claim 9, wherein contacting the vapor phase distillate with the plurality of adsorbent materials includes contacting the vapor phase distillate with the polymer resin before contacting the vapor phase distillate with the activated carbon.

17. A system for producing an ethanol product, the system comprising:
a vessel fluidly connected to a feed stream including ethanol at a concentration greater than 30% by volume, the vessel containing an adsorbent polymer resin, the adsorbent polymer resin including a polystyrene divinylbenzene copolymer, wherein a surface of the adsorbent polymer resin is hydrophobic.

18. The system of claim 17, wherein the feed stream is a liquid feed stream.

19. The system of claim 17, wherein the feed stream is a vapor phase feed stream.

20. The system of claim 19, further including a condenser fluidly coupled to the vessel.
